(19) United States
(12) Patent Application Publication
(10) Pub. No.: US 2017/0056345 A1
(43) Pub. Date: Mar. 2, 2017

(54) BOOSTER DRUG THERAPY FOR MYCOBACTERIUM INFECTIONS

(71) Applicant: STC.UNM, Albuquerque, NM (US)

(72) Inventors: Graham Timmins, Albuquerque, NM (US); Seong Won Choi, Albuquerque, NM (US)

(21) Appl. No.: 15/119,563

(22) PCT Filed: Feb. 18, 2015
(86) PCT No.: PCT/US2015/016359

§ 371 Date: Aug. 17, 2016

Related U.S. Application Data
(60) Provisional application No. 61/941,122, filed on Feb. 18, 2014.
(60) Provisional application No. 62/079,634, filed on Nov. 14, 2014.

(51) Int. Cl. A61K 31/16; A61K 9/00; A61K 45/06; A61K 31/4965

(52) U.S. Cl. CPC A61K 31/16; A61K 31/4965; A61K 9/0075; A61K 45/06

(57) ABSTRACT

In one embodiment, the invention provides a method of treating a subject who suffers from, or who is suspected of suffering from, a Mycobacterium infection, the method comprising administering to the subject a therapeutically effective amount of a urease inhibitor, optionally in combination with one or more anti-mycobacterial agents.

### CROSS-REFERENCE TO RELATED APPLICATIONS

This application claims priority from U.S. Provisional Patent Application No. 61/941,122, entitled “Method of Treating Mycobacteria”, and filed Feb. 18, 2014, and U.S. Provisional Patent Application No. 62/079,634, entitled “M. Tuberculosis Urease Inhibition to Treat TB and Enable Effective Immune Response”, and filed Nov. 14, 2014. The complete contents of each of these provisional patent applications are hereby incorporated by reference in their entirety.

### STATEMENT REGARDING FEDERAL FUNDING

This invention was not made with government support.

### FIELD OF THE INVENTION

In one embodiment, the invention provides a method of treating a subject suffering from a Mycobacterium infection (e.g. a Mycobacterium tuberculosis (Mtb) infection, a latent tuberculosis infection (LTBI) or a multidrug-resistant TB (MDR-TB) infection) by administering to the subject a therapeutically effective amount of a urease inhibitor (e.g. acetohydroxamic acid or a pharmaceutically acceptable salt or derivative thereof), optionally in combination with one or more anti-mycobacterial agents as described hereinafter.

In another embodiment, the invention provides a method of enhancing immunogenic protection from a vaccine against Mtb, the method comprising administering to a subject in need thereof a therapeutically effective amount of a urease inhibitor, optionally in combination with an additional anti-mycobacterial agent as described hereinafter.

Related pharmaceutical formulations are also provided.

### BACKGROUND OF THE INVENTION

Tuberculosis (TB) is an infectious disease caused by the bacillus Mycobacterium tuberculosis (Mtb). It typically affects the lungs (pulmonary TB) but can affect other sites as well (extrapulmonary TB). The disease is spread in the air when people who are sick with pulmonary TB expel bacteria, for example by coughing. Overall, a relatively small proportion of people infected with M. tuberculosis will develop TB disease. However, the probability of developing TB is much higher among people infected with HIV. TB is also more common among men than women, and affects mainly adults in the most economically productive age groups. WHO Global Tuberculosis Report 2014.

Tuberculosis ranks as the second leading cause of death from a single infectious agent, after the human immunodeficiency virus (HIV). Around 9 million people fell ill with TB in 2013, including 1.1 million cases among people living with HIV. In 2013, 1.5 million people died from TB, including 360 000 among people who were HIV-positive. Globally in 2013, an estimated 480,000 people developed multidrug-resistant TB (MDR-TB) and there were an estimated 210,000 deaths from MDR-TB. The number of people diagnosed with MDR-TB tripled between 2009 and 2013, and reached 136 000 worldwide. This was equivalent to 45% of the estimated MDR-TB cases among notified TB patients. Id.

Several antituberculosis compounds are bioprecursor prodrugs that require activation by Mycobacterium enzymes to acquire bacterial toxicity. These include pyrazinamide (PZA), isoniazid (INH) and ethionamide (ETA) (2-ethylthioiso-nicotinamide, 2-ethylpyrimidine-4-carbothioamide). PZA is activated by the mycobacterial pyrazinamidase (PncA) to pyrazinoic acid, which lowers the pH enhancing the intracellular accumulation of the latter. Pyrazinoic acid is unable to diffuse across the Mycobacterial cell wall, leading to the disruption of membrane transport and energy depletion. Because no pyrazinoic acid efflux mechanism exists, this accumulation process causes a remarkable susceptibility of M. tuberculosis to pyrazinamide. Mutations in the gene encoding pyrazinamidase/nicotinamidase (pncA) cause resistance to the antituberculosis drug pyrazinamide in tubercle bacillus. Both isoniazid and the structurally analogous thioamide, ethionamide, act as inhibitors of InhA (enoyl-acyl carrier protein reductase). However, the large majority of isoniazid-resistant strains remain full susceptible to ethionamide. This is due the fact that INH and ETA are activated by different mechanisms, thus avoiding cross-resistance. Chung, et al., Prodrugs for the Treatment of Neglected Diseases, Molecules 2008, 13, 616-677 (citations omitted).

Latent tuberculosis infection (LTBI) is a state of persistent immune response to stimulation by Mycobacterium tuberculosis antigens without evidence of clinically manifested active TB. A direct measurement tool for M. tuberculosis infection in humans is currently unavailable. WHO Latent Tuberculosis 2014. The following treatment regimens are currently recommended for LTBI: 6-month or 9-month isoniazid daily; 3-month rifapentine plus isoniazid weekly; 3-4 months isoniazid plus rifampicin daily; and 3-4 months rifampicin alone daily. Id.

Ethionamide has been used to treat MDR-TB; useful ethionamide dosages, however, can be hepatotoxic. Nitroimidazoles such as PA-824 (as a monotherapy or combined with moxifloxacin and pyrazinamide) and Delamanid have also been used in MDR-TB therapy. Bedaquiline (TMC207) is a Diarylquinoline which targets the adenosine triphosphate (ATP) synthase enzyme of the TB mycobacteria. Rifapentine and Delamanid are the subject of Phase 3 MDR-TB clinical trials.

The Mycobacterium tuberculosis gene EthR is a transcriptional regulator (repressor) controlling ethionamide bioactivation in Mycobacterium tuberculosis. Thiocarbamide-containing drugs, including ethionamide, are activated by the mycobacterial monooxygenase EthA, the production of which is controlled by EthR. Drug-like inhibitors of EthR boost the bioactivation of ethionamide. Willard, et al., “Synthetic EthR inhibitors boost antituberculosis activity of ethionamide”, Nat. Med. 2009 May; 15(5):537-44. See also J Med Chem. 2011 Apr. 28; 54(8):2994-3010; J Med Chem. 2012 Jan. 12; 55(1):68-83; and Acta Crystallogr C. 2013 November; 69(Pt 11):1243-50. The absence of boosters for isoniazid or pyrazinamide remains notable.

We discovered that isoniazid's anti-mycobacterial activity could be enhanced by selective stable isotopic substitution utilizing mass-independent isotope effects upon free radical intermediates in isoniazid activation. See U.S. Pat. No. 8,394,839.

However, the lack of radical intermediates in pyrazinamide activation precluded the use of mass-independent isotope effect approaches to enhance pyrazinamide's anti-mycobacterial activity.

Known vaccines against tuberculosis show very limited efficacy. The only available vaccine, Mycobacterium bovis BCG, is a highly attenuated live vaccine that exhibits limited efficacy and, due to its highly attenuated nature, proves ineffective in areas where prior environmental mycobacterial (EM) exposure has occurred. In contrast, virulent M. tuberculosis is highly infectious despite previous EM exposure. Notably, a previous M. tuberculosis infection, irrespective of treatment, does not protect against subsequent M. tuberculosis infection.

Urease is a mycobacterial enzyme that cleaves urea to CO₂ and ammonia; ammonia neutralizes acidic intracellular environments such as the phagosomes of macrophages in which Mtb can reside during infection (Clemens et al., J Bacti 1995). This neutralization leads to a decrease in phagosome-lysosome fusion, and inefficient antigen presentation, by attenuating MHC class II trafficking to the macrophage surface, a phenomenon which is reversed in a urease knockout mutant (Sendide et al. Infect. Immun. 2004). The importance of urease activity in immune responses is evidenced by the greater effectiveness of urease knockout recombinant BCG as a vaccine (Grode et al. J. Clin Invest. 2005), by better CD4⁺ T-cell responses (Mukai et al. FEMS Immunol. Med. Microbiol. 2008), and in better T cell activation in another recombinant strain of BCG (Tsukamoto et al. BMC Infect. Dis. 2014).

Although mycobacterial urease appears important in controlling immune responses, its role in virulence is unclear. Urease knock out bacteria readily infect mice, albeit with some slight inhibition of growth (Lin et al. Infect. Immun. 2012). Since urease appears unrelated to murine infection and is produced through an involved metabolism that includes acquisition of nickel, it is unclear why urease mutants are rarely isolated from human disease.

Thus, there is a continuing need for boosters of pyrazinamide anti-mycobacterial activity, especially those which minimize side effects in pyrazinamide-sensitive mycobacteria and/or enable the effective use of pyrazinamide against previously pyrazinamide-resistant mycobacteria. The increasing prevalence of MDR-TB (which can be defined as tuberculosis resistant to both isoniazid and rifampin) makes the need for pyrazinamide boosters particularly acute as around 36% to 85% multi-drug resistant tuberculosis patients exhibit pyrazinamide resistance. See Epidemiology of Pyrazinamide-Resistant Tuberculosis in the United States, 1999-2009, Clinical Infectious Disease (2013) 57: 1081-1093.

Further, utilizing urease inhibition to treat or inoculate against a M. tuberculosis infection would add substantially to the arsenal of effective mycobacterial disease treatments and satisfy a long-felt need for improved pharmacological treatment of MDR-TB and LTBI.

### SUMMARY OF THE INVENTION

We have discovered that a variety of mycobacterial infections, including MDR-TB, pyrazinamide-resistant TB and MDR-TB with pyrazinamide resistance, may be treated effectively by urease inhibitors including acetohydroxamic acid (lithostat) or a pharmaceutically acceptable salt or derivative thereof. Our use of pharmacological urease inhibitors stands in contrast to known genetic ablation of urease activity. In addition, it has been unexpectedly been discovered that acetohydroxamic acid itself exhibits significant anti-mycobacterial activity.

In one embodiment of our invention, a subject suffering from a Mycobacterium infection (e.g. a Mtb infection, LTBI or MDR-TB) is administered a therapeutically effective amount of a urease inhibitor such as acetohydroxamic acid or a pharmaceutically acceptable salt or derivative thereof, optionally in combination with one or more anti-mycobacterial agents such as pyrazinamide, pyrazinoic acid, isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin, among others and pharmaceutical salts/alternative pharmaceutical salts and mixtures thereof.

In a preferred embodiment, a subject suffering from a Mycobacterium infection is co-administered a therapeutically effective amount of acetohydroxamic acid and pyrazinamide and/or pyrazinoic acid, optionally in combination with one or more additional anti-mycobacterial agents as described above. In some embodiments, acetohydroxamic acid and optional additional anti-mycobacterial agents are administered orally, parenterally or by inhalation.

In another embodiment, the invention provides a pharmaceutical composition comprising a therapeutically effective amount of a urease inhibitor such as acetohydroxamic acid or a pharmaceutically acceptable salt or derivative thereof and one or more additional anti-mycobacterial agents such as pyrazinamide, pyrazinoic acid, isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin among others and pharmaceutically acceptable salts and mixtures thereof.

In still another embodiment, the invention provides a method of treating a subject who suffers from LTBI, the method comprising administering to the subject a therapeutically effective amount of a urease inhibitor such as acetohydroxamic acid or a pharmaceutically acceptable salt or derivative thereof, optionally in combination with one or more additional anti-mycobacterial agents such as pyrazinamide, pyrazinoic acid, isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin among others and pharmaceutically acceptable salts and mixtures thereof.

In still another embodiment, the invention provides a method of treating a subject who suffers from or who is at risk of developing a Mycobacterium infection (e.g. Mtb, MDR-TB, pyrazinamide-resistant TB or MDR-TB with pyrazinamide resistance), the method comprising administering to the subject a therapeutically effective amount of a urease inhibitor, optionally in combination with at least one additional anti-mycobacterial agent as described above, wherein in some embodiments, the additional anti-mycobacterial agent is other than pyrazinamide.

Pharmacological urease inhibitors such as acetohydroxamic acid and others as described herein can be administered prior to infection or immunogenic challenge, and in some embodiments urease inhibitors are administered concomitantly with a tuberculosis vaccine (e.g. BCG, recombinant BCG or another mycobacterial vaccine that does not incorporate urease knockouts or otherwise exhibit urease activity).

In certain embodiments, a urease inhibitor is co-administered with one or more antimycobacterial agents (e.g. anti-tuberculosis agents) selected from the group consisting of isoniazid, ethionamide, aminosalicyclic acid/aminosalicylate sodium, capreomycin sulfate, clofazimine, cycloserine, ethambutol hydrochloride (myambutol), kanamycin sulfate, pyrazinamide (which in certain embodiments is not used), rifabutin, rifampin, rifapentine, streptomycin sulfate, gatifloxacin and mixtures thereof, or pharmaceutically acceptable salts or alternative salts thereof.

In still another embodiment, the invention provides a method of enhancing immunogenic protection from a vaccine against M. tuberculosis, the method comprising administering to a subject in need thereof a therapeutically effective amount of a urease inhibitor, optionally in combination with an additional anti-mycobacterial agent as described above, wherein in certain instances the additional anti-mycobacterial agent is other than pyrazinamide.

In still another embodiment, the invention provides a method of treating a subject who suffers from a latent Mycobacterium infection (e.g. LTBI), the method comprising administering to the subject a therapeutically effective amount of a urease inhibitor, optionally in combination with an additional anti-mycobacterial agent as described herein (in certain embodiments the additional anti-mycobacterial agent is other than pyrazinamide), wherein administration of the urease inhibitor, optionally in combination with an additional anti-mycobacterial agent as described above, prevents the latent Mycobacterium infection from progressing to an active Mycobacterium infection.

While not wishing to be bound by any theory, we believe that urease inhibition reduces NH₃ and CO₂ levels in local environments like the phagosome which otherwise favor M. tuberculosis growth. Further, we theorize that urease inhibition upregulates levels of acidic phagosomes, thereby promoting phagosome-lysosome fusion, efficient antigen presentation of key antigens from the virulent Mtb and, in some cases, more effective T-cell response and greater infection control by host immunity. It is also believed that acetohydroxamic acid itself unexpectedly functions as an anti-mycobaterial agent in compositions and methods according to the present invention.

By successfully employing urease inhibition in mono- and co-therapies as described herein, our invention improves the prognosis of the large numbers of patients whose MDR-TB, pyrazinamide-resistant TB or MDR-TB with pyrazinamide resistance proved untreatable by conventional drug regimens. Enhancing the immunogenic protection of M. tuberculosis vaccines, and inhibiting a latent Mycobacterium infection from progressing to an active Mycobacterium infection, evince the significant clinical benefits realized by our invention.

These and other aspects of our invention are described further in the Detailed Description of the Invention.

### BRIEF DESCRIPTION OF THE FIGURES

FIG. 1 shows the incubation of M. bovis BCG in urease medium with increasing concentrations of acetohydroxamic acid. Inhibition of urease is evidenced by decreased red coloration.

FIG. 2 shows the treatment of M. tuberculosis with pyrazinamide (PZA) and acetohydroxamic acid (AHA) on solid media at pH 6 and at typical body fluid concentrations. The synergistic effect of AHA in overcoming urea protection of M. tuberculosis and in enhancing the efficacy of PZA is illustrated.

### DETAILED DESCRIPTION OF THE INVENTION

In accordance with the present invention there may be employed conventional chemical synthetic methods and other biological and pharmaceutical techniques within the skill of the art. Such techniques are well-known and are otherwise explained fully in the literature.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It is to be noted that as used herein and in the appended claims, the singular forms “a,” “and” and “the” include plural references unless the context clearly dictates otherwise.

Furthermore, the following terms shall have the definitions set out below. It is understood that in the event a specific term is not defined hereinbelow, that term shall have a meaning within its typical use within context by those of ordinary skill in the art.

The term “compound”, as used herein, unless otherwise indicated, refers to any specific chemical compound disclosed herein. Within its use in context, the term generally refers to a single compound, i.e., a small molecule. In certain instances the term may also refer to stereoisomers and/or optical isomers (including racemic mixtures) or enantiomerically enriched mixtures of disclosed compounds. Compounds which are disclosed are those which are stable and where a choice of substituents and claim elements is available, the substituent or claim element is chosen such that stable compounds are formed from the disclosed elements and substituents. The symbol [symbol] in a chemical structure or formula signifies that either a double or single bond may be present between the atoms to which such symbol is attached, depending upon the valence of those atoms and substituents which are on such atoms.

The term “patient” or “subject” is used throughout the specification within context to describe an animal, especially including a domesticated mammal (i.e., other than a laboratory test animal such as a dog, cat, cow, horse, sheep, goat, etc.) and preferably a human, to whom a treatment or procedure, including a prophylactic treatment or procedure is performed. For treatment of those infections, conditions or disease states which are specific for a specific animal such as a human patient, the term patient refers to that specific animal. In most instances, the patient or subject of the present invention is a domesticated/agricultural animal or human patient of either or both genders.

The term “effective” is used herein, unless otherwise indicated, to describe an amount of a compound or composition which, in context, is used to produce or effect an intended result, whether that result relates to the treatment of a mycobacterial infection in a patient or subject or another intended effect. The term effective subsumes all other effective amount or effective concentration terms which are otherwise described or used in the present application.

“Hydrocarbon” or “hydrocarbyl” refers to any monovalent (or divalent in the case of alkylene groups) radical containing carbon and hydrogen, which may be straight, branch-chained or cyclic in nature. Hydrocarbons include linear, branched and cyclic hydrocarbons, including alkyl groups, alkylene groups, saturated and unsaturated hydrocarbon groups including aromatic groups both substituted and unsubstituted, alkene groups (containing double bonds between two carbon atoms) and alkyne groups (containing triple bonds between two carbon atoms). In certain instances, the terms substituted alkyl and alkylene are sometimes used synonymously.

“Alkyl” refers to a fully saturated monovalent radical containing carbon and hydrogen, and which may be cyclic, branched or a straight chain. Examples of alkyl groups are methyl, ethyl, n-butyl, n-hexyl, n-heptyl, n-octyl, n-nonyl, n-decyl, isopropyl, 2-methyl-propyl, cyclopropyl, cyclopropylmethyl, cyclobutyl, cyclopentyl, cyclopentylethyl, cyclohexylethyl and cyclohexyl. Preferred alkyl groups are C₁-C₆ alkyl groups. “Alkylene” refers to a fully saturated hydrocarbon which is divalent (may be linear, branched or cyclic) and which is optionally substituted. Preferred alkylene groups are C₁-C₆ alkylene groups. Other terms used to indicate substitutent groups in compounds according to the present invention are as conventionally used in the art.

The term “aryl” or “aromatic”, in context, refers to a substituted or unsubstituted monovalent aromatic radical having a single ring (e.g., benzene or phenyl). Other examples of aryl groups, in context, may include heterocyclic aromatic ring systems “heteroaryl” groups having one or more nitrogen, oxygen, or sulfur atoms in the ring (5- or 6-membered heterocyclic rings) such as imidazole, furyl, pyrrole, pyridyl, furanyl, thiene, thiazole, pyridine, pyrimidine, pyrazine, triazole, oxazole, among others, which may be substituted or unsubstituted as otherwise described herein.

The term “heterocyclic group” “heterocycle” as used throughout the present specification refers to an aromatic (“heteroaryl”) or non-aromatic cyclic group forming the cyclic ring and including at least one and up to three hetero atoms such as nitrogen, sulfur or oxygen among the atoms forming the cyclic ring. The heterocyclic ring may be saturated (heterocyclic) or unsaturated (heteroaryl). Exemplary heterocyclic groups include, for example pyrrolidinyl, piperidinyl, morpholinyl, pyrrole, pyridine, pyridone, pyrimidine, imidazole, thiophene, furan, pyran, thiazole, more preferably pyrimidinyl, pyrrolidinyl, piperidinyl, morpholinyl, oxazole, isoxazole, pyrrole, pyridine, thiophene, thiazole and even more preferably pyrimidinyl, especially uracil or cytosine which are optionally substituted, furyl, 3-methylfuryl, thiazole, piperazinyl, N-methylpiperazinyl, tetrahydropyranyl and 1,4-dioxane, among others. Additional heterocyclic groups include oxazole, benzoxazole, pyrrole, dihydropyrrole, benzopyrrole, benzodihydropyrrole, indole, indolizine, among others.

Exemplary heteroaryl moieties which may be used in the present invention include for example, pyrrole, pyridine, pyridone, pyridazine, pyrimidine, pyrazine, pyrazole, imidazole, triazole, tetrazole, oxadiazole, sulfur-containing aromatic heterocycles such as thiophene; oxygen-containing aromatic heterocycles such as furan and pyran, and including aromatic heterocycles comprising 2 or more hetero atoms selected from among nitrogen, sulfur and oxygen, such as thiazole, thiadiazole, isothiazole, isoxazole, furazan and oxazole. Further heteroaryl groups may include pyridine, triazine, pyridone, pyrimidine, imidazole, furan, pyran, thiazole. Pyrimidine groups, especially uracil and cytosine, optionally substituted, are preferred.

The term “substituted” shall mean substituted at a carbon (or nitrogen) position within context, hydroxyl, carboxyl, cyano (C≡N), nitro (NO₂), halogen (preferably, 1, 2 or 3 halogens, especially on an alkyl, especially a methyl group such as a trifluoromethyl), alkyl group (preferably, C₁-C₁₀, more preferably, C₁-C₆), alkoxy group (preferably, C₁-C₆ alkyl or aryl, including phenyl and substituted phenyl), ester (preferably, C₁-C₆ alkyl or aryl) including alkylene ester (such that attachment is on the alkylene group, rather than at the ester function which is preferably substituted with a C₁-C₆ alkyl or aryl group), preferably, C₁-C₆ alkyl or aryl, halogen (preferably, F or Cl), nitro or amine (including a five- or six-membered cyclic alkylene amine, further including a C₁-C₆ alkyl amine or C₁-C₆ dialkyl amine which alkyl groups may be substituted with one or two hydroxyl groups), amido, which is preferably substituted with one or two C₁-C₆ alkyl groups (including a carboxamide which is substituted with one or two C₁-C₆ alkyl groups), alkanol (preferably, C₁-C₆ alkyl or aryl), or alkanoic acid (preferably, C₁-C₆ alkyl or aryl). Preferably, the term “substituted” shall mean within its context of use alkyl, alkoxy, halogen, ester, keto, nitro, cyano and amine (especially including mono- or di-C₁-C₆ alkyl substituted amines which may be optionally substituted with one or two hydroxyl groups). Any substitutable position in a compound according to the present invention may be substituted in the present invention, but no more than 3, more preferably no more than 2 substituents (in some instances only 1 or no substituents) is present on a ring. Preferably, the term “unsubstituted” shall mean substituted with one or more H atoms.

“Halogen” or “halo” may be fluoro, chloro, bromo or iodo.

A “hydrolyzable moiety” can be methyl, t-butyl, benzyl, p-methoxybenzyl, p-nitrobenzyl, allyl, trityl, methoxymethyl, 2-methoxypropyl, methoxyethoxymethyl, ethoxyethyl, tetrahydropyranyl, tetrahydrothiopyranyl, and trialkylsilyl ethers such as trimethylsilyl ether, triethylsilyl ether, dimethylarylsilyl ether, triisopropylsilyl ether and t-butyldimethylsilyl ether; esters such as benzoyl, acetyl, phenylacetyl, formyl, mono-, di-, and trihaloacetyl such as chloroacetyl, dichloroacetyl, trichloroacetyl, trifluoroacetyl; and carbonates including but not limited to alkyl carbonates having from one to six carbon atoms such as methyl, ethyl, n-propyl, isopropyl, n-butyl, t-butyl; isobutyl, and n-pentyl; alkyl carbonates having from one to six carbon atoms and substituted with one or more halogen atoms such as 2,2,2-trichloroethoxymethyl and 2,2,2-trichloro-ethyl; alkenyl carbonates having from two to six carbon atoms such as vinyl and allyl; cycloalkyl carbonates having from three to six carbon atoms such as cyclopropyl, cyclobutyl, cyclopentyl and cyclohexyl; and phenyl or benzyl carbonates optionally substituted on the ring with one or more C₁₋₆ alkoxy, or nitro.

The term “co-administration” is used to describe the administration of two active compounds. Although the term co-administration preferably includes the administration of two active compounds to the patient at the same time, it is not necessary that the compounds actually be administered at the exact same time, only that amounts of compound will be administered to a patient or subject such that effective concentrations are found in the blood, serum or plasma, or in the pulmonary tissue at the same time. In the present invention, the term co-administration refers to the administration of a urease inhibitor in combination with an anti-tuberculosis agent or the administration of a urease inhibitor with a tuberculosis vaccine or during the period when the patient or subject is developing immunity to M. tuberculosis as a consequence of vaccine administration or immunogenic challenge.“Mycobacterium infections” are infections caused by intracellular microorganisms of the genus Mycobacterium, including diseases caused by the species M. tuberculosis, M. africanum, M. bovis, M. bovis BCG, M. canetti, M. microti, M. caprae, M. pinnipedii, M. avium, and M. leprae. “Mycobacterium infections” include infections caused by members of the Mycobacterium tuberculosis complex, the Mycobacterium avium complex, the Mycobacterium gordonae clade, the Mycobacterium kansasii clade, the Mycobacterium nonchromogenicum/terrae clade, the Mycolactone-producing mycobacteria, the Mycobacterium simiae clade, the Mycobacterium chelonae clade, the Mycobacterium fortuitum clade, the Mycobacterium parafortuitum clade and the Mycobacterium vaccae clade.

“Mycobacterium infections” include infections associated with nontuberculosis mycobacteria (NTM), which are classified based on their growth rates. Rapidly growing NTM are categorized into pigmented and nonpigmented species. Mycobacterium fortuitum complex is nonpigmented and includes the M. fortuitum group and the Mycobacterium chelonae/abscessus group. The pigmented species are rarely associated in clinical disease and include Mycobacterium phlei, Mycobacterium aurum, Mycobacterium flavescens, Mycobacterium vaccae, Mycobacterium neoaurum, and Mycobacterium thermoresistible. Mycobacterium smegmatis may be either pigmented or nonpigmented.

“Mycobacterium infections” also include atypical mycobacterial infections. Mycobacterium avium complex (MAC) and Mycobacterium scrofulaceum are associated with lymphadenitis in immunocompetent children. MAC has also been associated with the pulmonary infection and bronchiectasis in elderly women without a preexisting lung disease. Pulmonary MAC infection in this population is believed to be due to voluntary cough suppression that results in stagnation of secretions, which is suitable for growth of the organisms. Mycobacterium ulcerans, the agent of a chronic ulcerative skin infection called Buruli ulcer, is widespread in Ghana, Cote d'Ivoire, Senegal, Uganda, and most central African countries. Medscape, Atypical Mycobacterial Infection.

The term “Tuberculosis” or “TB” is used to describe the infection caused by the infective agent “Mycobacterium tuberculosis” or “M. tuberculosis”, a tubercle bacillus bacteria. Tuberculosis is a potentially fatal contagious disease that can affect almost any part of the body but is most frequently an infection of the lungs. It is caused by a bacterial microorganism, the tubercle bacillus or Mycobacterium tuberculosis.

Tuberculosis is primarily an infection of the lungs, but any organ system is susceptible, so its manifestations may be varied. Effective therapy and methods of control and prevention of tuberculosis have been developed, but the disease remains a major cause of mortality and morbidity throughout the world. The treatment of tuberculosis has been complicated by the emergence of drug-resistant organisms, including multiple-drug-resistant tuberculosis, especially in those with HIV infection.

Mycobacterium tuberculosis, the causative agent of tuberculosis, is transmitted by airborne droplet nuclei produced when an individual with active disease coughs, speaks, or sneezes. When inhaled, the droplet nuclei reach the alveoli of the lung. In susceptible individuals the organisms may then multiply and spread through lymphatics to the lymph nodes, and through the bloodstream to other sites such as the lung apices, bone marrow, kidneys, and meninges.

The development of acquired immunity in 2 to 10 weeks results in a halt to bacterial multiplication. Lesions heal and the individual remains asymptomatic. Such an individual is said to have tuberculosis infection without disease, and will show a positive tuberculin test. The risk of developing active disease with clinical symptoms and positive cultures for the tubercle bacillus diminishes with time and may never occur, but is a lifelong risk. Approximately 5% of individuals with tuberculosis infection progress to active disease. Progression occurs mainly in the first 2 years after infection; household contacts and the newly infected are thus at risk.

Many of the symptoms of tuberculosis, whether pulmonary disease or extrapulmonary disease, are nonspecific. Fatigue or tiredness, weight loss, fever, and loss of appetite may be present for months. A fever of unknown origin may be the sole indication of tuberculosis, or an individual may have an acute influenza-like illness. Erythema nodosum, a skin lesion, is occasionally associated with the disease.

The lung is the most common location for a focus of infection to flare into active disease with the acceleration of the growth of organisms. Infections in the lung are the primary focus of the present invention. There may be complaints of cough, which can produce sputum containing mucus, pus- and, rarely, blood. Listening to the lungs may disclose rales or crackles and signs of pleural effusion (the escape of fluid into the lungs) or consolidation if present. In many, especially those with small infiltration, the physical examination of the chest reveals no abnormalities.

Miliary tuberculosis is a variant that results from the blood-borne dissemination of a great number of organisms resulting in the simultaneous seeding of many organ systems. The meninges, liver, bone marrow, spleen, and genitourinary system are usually involved. The term miliary refers to the lung lesions being the size of millet seeds (about 0.08 in. or 2 mm). These lung lesions are present bilaterally. Symptoms are variable.

Extrapulmonary tuberculosis is much less common than pulmonary disease. However, in individuals with AIDS, extrapulmonary tuberculosis predominates, particularly with lymph node involvement, with some pulmonary impact. For example, fluid in the lungs and lung lesions are other common manifestations of tuberculosis in AIDS. The lung is the portal of entry, and an extrapulmonary focus, seeded at the time of infection, breaks down with disease occurring.

Development of renal tuberculosis can result in symptoms of burning on urination, and blood and white cells in the urine; or the individual may be asymptomatic. The symptoms of tuberculosis meningitis are nonspecific, with acute or chronic fever, headache, irritability, and malaise.

A tuberculosis pleural effusion can occur without obvious lung involvement. Fever and chest pain upon breathing are common symptoms. Bone and joint involvement result in pain and fever at the joint site. The most common complaint is a chronic arthritis usually localized to one joint. Osteomyelitis is also usually present. Pericardial inflammation with fluid accumulation or constriction of the heart chambers secondary to pericardial scarring are two other forms of extrapulmonary disease.

At present, the principal methods of diagnosis for pulmonary tuberculosis are the tuberculin skin test (an intracutaneous injection of purified protein derivative tuberculin is performed, and the injection site examined for reactivity), sputum smear and culture, and the chest x-ray. Culture and biopsy are important in making the diagnosis in extrapulmonary disease.

A combination of two or more drugs is often used in the initial therapy of tuberculosis disease. Drug combinations are used to lessen the chance of drug-resistant organisms surviving. The preferred treatment regimen for both pulmonary and extrapulmonary tuberculosis is a 6-month regimen of the antibiotics isoniazid, rifampin, and pyrazinamide for 2 months, followed by isoniazid and rifampin for 4 months. Increasingly ethionamide/prothionamide is being used in place of pyrazinamide or one of the other anti-mycobacterial drugs in the treatment of tuberculosis, especially drug resistant disease. Accordingly, determining the ethionamide/prothionamide resistance of the M. tuberculosis infection is becoming an increasingly important feature in tuberculosis therapy. Because of the problem of drug-resistant cases, ethambutol or other drugs can be included in the initial regimen until the results of drug susceptibility studies are known. Once treatment is started, improvement occurs in almost all individuals. Any treatment failure or individual relapse is usually due to drug-resistant organisms.

Bacille Calmette-Guerin (BCG), a live and attenuated strain of Mycobacterium bovis, is the only available vaccine against TB and has been used for the vaccination of newborns for decades. This vaccine has its limitations however, and progress in generating more effective TB vaccines has been made with several candidate vaccines in recent years. One candidate that has been in clinical trials is based on adenovirus serotype 35 expressing Ag85A, Ag85B and TB10.4 antigens of Mtb. This vaccine has been demonstrated to be safe in uninfected people and was able to induce high T-cell responses against Mtb antigens of the vaccine, making it a promising candidate for a prophylactic TB vaccine.” U.S. Pat. No. 8,771,709 (citations omitted).

As summarized in Montagnan, et al., “Vaccine against tuberculosis: what's new?”, BMC Infectious Diseases 2014, 14(Suppl 1):S2, Mtb candidate vaccines include the following. “MVA85A . . . a recombinant strain of Modified Vaccinia virus Ankara expressing the Mtb antigen 85A (Ag85A), designed to enhance response induced by BCG. AdAg85a is a recombinant strain of replication-deficient adenoviral vector expressing the MtbAg85A. Ad35/Aeras 402 is a recombinant, non-replicating adenovirus, serotype 35 vaccine, which expresses a fusion protein from the Mtb Ag85A, antigens 85B (Ag85B) and TB10.4. H1/IC31 is a recombinant subunit vaccine, composed by the hybrid protein of Early Secretory Antigenic Target 6 (ESAT6) and Ag85B adjuvanted with IC31, an adjuvant system composed by the cationic protein polyaminoacid KLK and oligodeoxynucleotide ODN1a. HyVac4/Aeras 404 is a booster vaccine developed by the same group of H1/IC31. The antigen ESAT6 was replaced by TB10.4, to avoid the interference with IGRAs and the fusion protein was combined with the adjuvant IC31. ID93/GLA-SE is a protein-adjuvant vaccine, composed by ID93, a fusion protein comprising four Mtb antigens (Rv2608, Rv3619, Rv3620 and Rv1813), combined with the glucopyranosyl lipid adjuvant-stable emulsion (GLA-SE). H56/IC31 is a protein-adjuvant vaccine composed by H56, a fusion protein containing Ag85B, ESAT6 and the latency-associated protein Rv2660c, combined with the adjuvant IC31. M72/AS01E is a recombinant vaccine developed to boost BCG-induced or Mtb-induced immune response. VPM1002 is a recombinant BCG strain that expresses membrane-perforating listeriolysin (encoded by the gene hly) of Listeria monocytogenes, lacking the urease C gene (BCG ΔureC::hly) and that contains a hygromycin resistance marker. MTBVAC is the first live-attenuated Mtb vaccine entered in phase 1 clinical trial in January 2013. It derives from the SO2, an attenuated strain obtained by the insertion of a kanamycin-resistance cassette in the phoP gene of Mtb. phoP is a transcription regulator, therefore its mutation determines lack of expression of several genes, including virulence factors, such as ESAT6. RUTI® is a therapeutic vaccine constituted by detoxified liposomal fragments of Mtb. A whole inactivated Mycobacterium vaccae (MV) administered intradermally was firstly evaluated as a therapeutic vaccine.”

“Acetohydroxamic acid” (AHA or Lithostat) is a potent and irreversible inhibitor of bacterial and plant urease and is usually used to treat urinary tract infections. Acetohydroxamic acid is similar to urea, but is not hydrolyzable by the urease enzyme. W. Fishbein and P. Carbone J Biol Chem. 1965 June; 240:2407-14. “Acetohydroxamic acid derivatives and pharmaceutically acceptable salts” include, but are not limited to the compounds disclosed in U.S. Pat. No. 4,183,951 (e.g. 1-Adamantyl-carbamoyl-acetohydroxamic acid, α-Naphthylcarbamoyl-acetohydroxamic acid, 4-Chlorophenylcarbamoyl-acetohydroxamic acid and its metal salts, 4-Aminophenylcarbamoyl-acetohydroxamic acid and its metal and acid addition salts, 3,4-Dichlorophenyl-carbamoyl-acetohydroxamic acid and its metal salts, 2,6-Dichlorophenylcarbamoyl-acetohydroxamic acid and its metal salts, 3,4,5-Trimethoxyphenylcarbamoyl-acetohydroxamic acid and its metal salts, 3-Trifluoromethylphenylcarbamoylacetohydroxamic acid and its metal salts, phenylureido-acetohydroxamic acid and its metal salts, p-Chlorophenyl-ureido-acetohydroxamic acid and its metal salts and N,N-Diphenylureido-acetohydroxamic acid and its metal salts.

“Urease inhibitors” include acetohydroxamic acid derivatives and pharmaceutically acceptable salts as described herein, as well as compositions which include, but which are not limited to compounds of Formula (I):

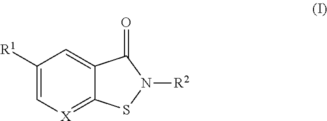

wherein R¹ represents a hydrogen atom or an amino group, R² represents a hydrogen atom, a lower alkyl group, or an acetyl group, and X represents a carbon atom or a nitrogen atom, including 1,2-benzoisothiazol-3(2H)-one, isothiazolo[5,4-b]pyridin-3(2H)-one, 5-amino-1,2-benzoisothiazol-3(2H)-one, N-methyl-1,2-benzoisothiazol-3(2H)-one and N-acetyl-1,2-benzoisothiazol-3(2H)-one (see U.S. Patent Application Document No. 20040058952), pentacyclic triterpenoid acids isolated from Boswellia carterii such as 3-O-acetyl-9,11-dehydro-β-boswellic acid; 3-O-acetyl-11-hydroxy-β-boswellic acid; 3-O-acetyl-11-keto-β-boswellic acid and 11-keto-β-boswellic acid, caprylohydroxamic acid, herbal methanolic extracts of Matricaria disciforme, Nasturtium officinale, Punica granatum, Camelia sinensis, and Citrus aurantifolia, bismuth complexes such as Bi(EDTA), Bi(Cys)₃ and ranitidine bismuth citrate (RBC), hydroxyurea and thiourea, monastrol, quercetin, DL-phenylalanine hydroxamic acid, fluorofamide, (+) catechin hydrate, (−)epigallocatechin gallate, fluoroamide, Baicalin, scutellarin, bis(aminomethyl)phosphinic acid and its derivatives, aminomethyl (N-n-hexylaminomethyl)phosphinic acid, urea and thiourea derivatives of the conjugates of Gly, Pro, Phe, Glu(OBzl), Tyr(2, 6-Cl₂-Bzl) and Lys(Z) as described in the Int'l. J. Chem. and Pharm. Sci. September 2013, 4(3).

In methods of treatment of the invention, a urease inhibitor may be administered either alone or preferably in combination with other antimycobacterial agents (e.g. anti-tuberculosis agents) including isoniazid, ethionamide, aminosalicyclic acid/aminosalicylate sodium, capreomycin sulfate, clofazimine, cycloserine, ethambutol hydrochloride (myambutol), kanamycin sulfate, pyrazinamide (which in certain embodiments is not used), rifabutin, rifampin, rifapentine, streptomycin sulfate, gatifloxacin and mixtures thereof. In some embodiments, these additional anti-tuberculosis agents are labeled isotopically, e.g. ¹⁵N-ethionamide; ³³S-ethionamide, ³⁴S-ethionamide and ³⁶S-ethionamide; and ¹⁵N-isoniazid, as described here.

In addition, the urease inhibitor may be particularly effective as a booster of pyrazinamide and/or as a compound exhibiting its own antimycobacterial activity. By inhibiting urease, using an agent such as acetohydroxamic acid (lithostat), the anti-tuberculosis therapy becomes far more effective resulting in a far higher rate of favorable therapy and including a cure of tuberculosis.

In certain embodiments of treating tuberculosis infections in accordance with the methods of treatment of the invention, a urease inhibitor may be administered either alone or in combination with drug regimens that do not include pyrazinamide. These agents include isoniazid, ethionamide, aminosalicyclic acid/aminosalicylate sodium, capreomycin sulfate, clofazimine, cycloserine, ethambutol hydrochloride (myambutol), kanamycin sulfate, rifabutin, rifampin, rifapentine, streptomycin sulfate, gatifloxacin and mixtures thereof. Such treatments may inhibit mycobacterial urease prior to or during TB drug regimens in infected patients to enable a more effective immune response and to enhance drug therapy in drug sensitive and resistant strains.

Or such methods of treatment may be administered as part of vaccination strategies that use BCG, recombinant BCG or other mycobacterial vaccines that lack urease knockouts and exhibit urease activity.

In certain embodiments, a urease inhibitor may be co-administered with an anti-mycobacterial compound of the Formula (II):

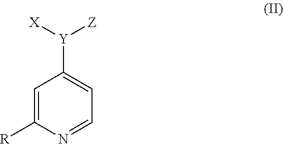

where X is an oxygen atom selected from the group consisting of ¹⁷O and ¹⁸O; Y is a carbon atom selected from the group consisting of ¹²C and ³³C; Z is a NHNH₂ group, which group is optionally isotopically labeled with at least one ¹⁵N atom; and R is H. See U.S. Pat. No. 8,921,569.

Although the compositions described herein may be administered by any route of administration, including parenteral, topical or oral administration among others, in preferred aspects of the invention, the urease inhibitor is administered to the lungs of the subject via pulmonary administration, including intratracheal administration. The pharmaceutical composition of the invention for pulmonary administration is usually used as an inhalant. The composition can be formed into dry powder inhalants, inhalant suspensions, inhalant solutions, encapsulated inhalants and like known forms of inhalants. Such forms of inhalants can be prepared by filling the pharmaceutical composition of the invention into an appropriate inhaler such as a metered-dose inhaler, dry powder inhaler, atomizer bottle, nebulizer etc. before use. Of the above forms of inhalants, powder inhalants may be preferable.

When the pharmaceutical composition of the invention is used in the form of a powder, the mean particle diameter of the powder is not especially limited but, in view of the residence of the particles in the lungs, is preferably that the particles fall within the range of about 0.1 to 20 μm, and particularly about 1 to 5 μm. Although the particle size distribution of the powder pharmaceutical composition of the invention is not particularly limited, it is preferable that particles having a size of about 25 μm or more account for not more than about 5% of the particles, and preferably, 1% or less to maximize delivery into the lungs of the subject.

The pharmaceutical composition in the form of a powder of the invention can be produced by, for example, using the drying-micronization method, the spray drying method and standard pharmaceutical methodology well known in the art.

By way of example without limitation, according to the drying-pulverization method, the pharmaceutical composition in the form of a powder can be prepared by drying an aqueous solution (or aqueous dispersion) containing the active(s) and excipients which provide for immediate release in pulmonary tissue and microparticulating the dried product. Stated more specifically, after dissolving (or dispersing) a pharmaceutically acceptable carrier, additive or excipient in an aqueous medium, the active(s) in effective amount is added and dissolved (or dispersed) by stirring using a homogenizer, etc. to give an aqueous solution (or aqueous dispersion). The aqueous medium may be water alone or a mixture of water and a lower alcohol. Examples of usable lower alcohols include methanol, ethanol, 1-propanol, 2-propanol and like water-miscible alcohols. Ethanol is particularly preferable. After the obtained aqueous solution (or aqueous dispersion) is dried by blower, lyophilization, etc., the resulting product is pulverized or microparticulated into fine particles using jet mills, ball mills or like devices to give a powder having the above mean particle diameter. If necessary, additives as mentioned above may be added in any of the above steps.

According to the spray-drying method, the pharmaceutical composition in the form of a powder of the invention can be prepared, for example, by spray-drying an aqueous solution (or aqueous dispersion) containing ethionamide/prothionamide and excipients, additives or carriers for microparticulation. The aqueous solution (or aqueous dispersion) can be prepared following the procedure of the above drying-micronization method. The spray-drying process can be performed using a known method, thereby giving a powdery pharmaceutical composition in the form of globular particles with the above-mentioned mean particle diameter.

The inhalant suspensions, inhalant solutions, encapsulated inhalants, etc. can also be prepared using the pharmaceutical composition in the form of a powder produced by the drying-micronization method, the spray-drying method and the like, or by using a carrier, additive or excipient and ethionamide/prothionamide that can be administered via the lungs, according to known preparation methods.

Furthermore, the inhalant comprising the pharmaceutical composition of the invention is preferably used as an aerosol. The aerosol can be prepared, for example, by filling the pharmaceutical composition of the invention and a propellant into an aerosol container. If necessary, dispersants, solvents and the like may be added. The aerosols may be prepared as 2-phase systems, 3-phase systems and diaphragm systems (double containers). The aerosol can be used in any form of a powder, suspension, solution or the like.

Examples of usable propellants include liquefied gas propellants, compressed gases and the like. Usable liquefied gas propellants include, for example, fluorinated hydrocarbons (e.g., CFC substitutes such as HCFC-22, HCFC-123, HFC-134a, HFC-227 and the like), liquefied petroleum, dimethyl ether and the like. Usable compressed gases include, for example, soluble gases (e.g., carbon dioxide, nitric oxide), insoluble gases (e.g., nitrogen) and the like.

The dispersant and solvent may be suitably selected from the additives mentioned above. The aerosol can be prepared, for example, by a known 2-step method comprising the step of preparing the composition of the invention and the step of filling and sealing the composition and propellant into the aerosol container.

As a preferred embodiment of the aerosol according to the invention, the following aerosol can be mentioned: Examples of the compounds to be used include isotopically labeled isoniazid, isotopically labeled urea or mixtures thereof. As propellants, fluorinated hydrocarbons such as HFC-134a, HFC-227 and like CFC substitutes are preferable. Examples of usable solvents include water, ethanol, 2-propanol and the like. Water and ethanol are particularly preferable. In particular, a weight ratio of water to ethanol in the range of about 0:1 to 10:1 may be used.

The aerosol of the invention contains excipient in an amount ranging from about 0.01 to about 10⁴ wt. % (preferably about 0.1 to 10³ wt. %), propellant in an amount of about 10² to 10⁷ wt. % (preferably about 10³ to 10⁶ wt. %), solvent in an amount of about 0 to 10⁶ wt. % (preferably about 10 to 10⁵ wt. %), and dispersant in an amount of 0 to 10³ wt. % (preferably about 0.01 to 10² wt. %), relative to the weight of isoniazid and/or urea which is included in the final composition.

The pharmaceutical compositions of the invention are safe and effective for use in the treatment or prevention (reducing the likelihood) of a M. tuberculosis infection according to the present invention. Although the dosage of the composition of the invention may vary depending on the type of active substance administered as well as the nature (size, weight, etc.) of the subject to be treated, the composition is administered in an amount effective for allowing the pharmacologically active substance to be effective. For example, the composition is preferably administered such that the active ingredient can be given to a human adult in a dose of about 0.001 to about 750 mg or more, about 0.01 mg to about 500 mg, about 0.05 mg to about 400 mg, about 0.1 mg to about 350 mg, about 0.5 mg to about 300 mg, about 1 to about 250 mg.

The amount of a urease inhibitor that may be combined with the carrier materials to produce a single dosage form will vary depending upon the host treated and the particular mode of administration. Preferably, the compositions should be formulated so that a therapeutically effective dosage of between about 1 and 25 mg/kg, about 5 to about 15 mg/kg of patient/day of the urease inhibitor can be administered to a patient receiving these compositions. Preferably, pharmaceutical compositions in dosage form according to the present invention comprise a therapeutically effective amount of at least about 10 mg of a urease inhibitor, at least about 25 mg of urease inhibitor, at least 50 mg of a urease inhibitor, at least 60 mg of a urease inhibitor, at least 75 mg of a urease inhibitor, at least 100 mg of a urease inhibitor, at least 150 mg of a urease inhibitor, at least 200 mg of a urease inhibitor, at least 250 mg of a urease inhibitor, at least 300 mg of a urease inhibitor, about 350 mg of a urease inhibitor, about 400 mg of a urease inhibitor, about 500 mg of a urease inhibitor, about 750 mg of a urease inhibitor, about 1 g (1,000 mg) of a urease inhibitor, alone or in combination with a therapeutically effective amount of at least one additional anti-tuberculosis agent, especially pyrazinamide and/or pyrazinoic acid. Exemplary additional anti-tuberculosis agents which may be used in pharmaceutical compositions include one or more of isoniazid, ethionamide, aminosalicyclic acid/aminosalicylate sodium, capreomycin sulfate, clofazimine, cycloserine, ethambutol hydrochloride (myambutol), kanamycin sulfate, rifabutin, rifampin, rifapentine, streptomycin sulfate, gatifloxacin or pharmaceutically acceptable salts or alternative salts and mixtures thereof, all in therapeutically effective amounts.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

The form of the pharmaceutical composition of the invention such as a powder, solution, suspension etc. may be suitably selected according to the type of substance to be administered.

As an administration route, direct inhalation via the mouth using an inhaler is usually preferable. Since the pharmaceutical composition of the invention allows direct local administration into the airways and in particular, directly to pulmonary tissue, the active substance contained therein produces immediate effects. Furthermore, the composition is formulated as an immediate release product so that cleavage and analysis can begin soon after administration.

The invention is illustrated further in the following non-limiting examples.

### Example 1

### Acetohydroxamic Acid Inhibits Mycobacterial Urease

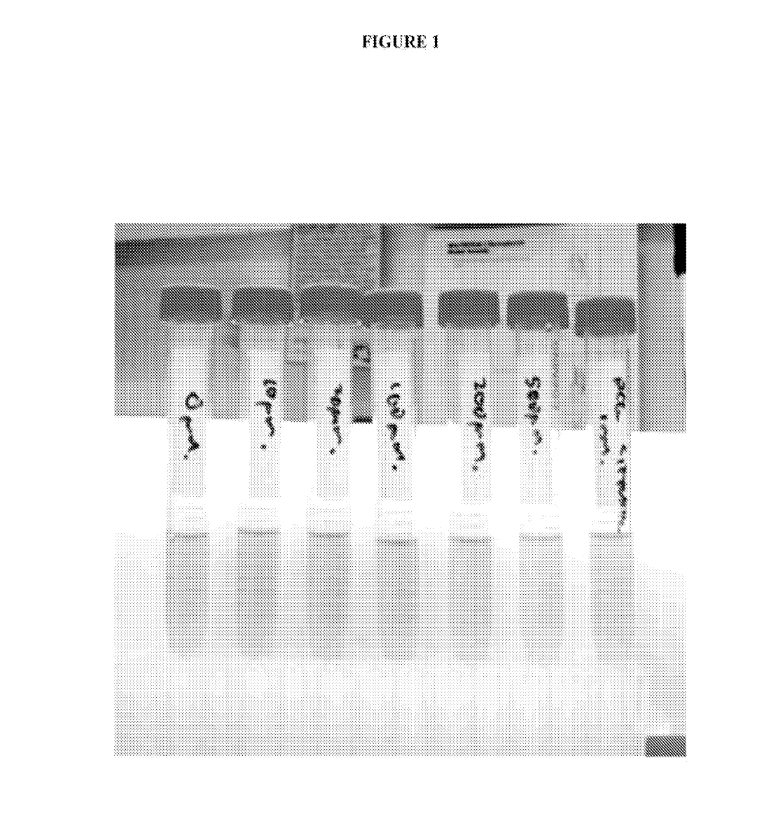

In our assessment of the effect of acetohydroxamic acid upon mycobacteria, we determined that acetohydroxamic acid inhibited urease activity in a whole-cell assay of mycobacteria with an IC₅₀ of approximately 100 to 200 μM (FIG. 1). We attributed the pyrazinamide boosting effect of acetohydroxamic acid to its ability to inhibit mycobacterial urease. FIG. 1 shows the incubation of M. bovis BCG in urease medium with increasing concentrations of acetohydroxamic acid. Inhibition of urease is evidenced by decreased red coloration. It is noted that the formation of acetohydroxamic acid resulted in an unexpected enhanced antimicrobial activity.

### Example 2

### AHA is a PZA Booster and an Anti-Mycobacterial Agent

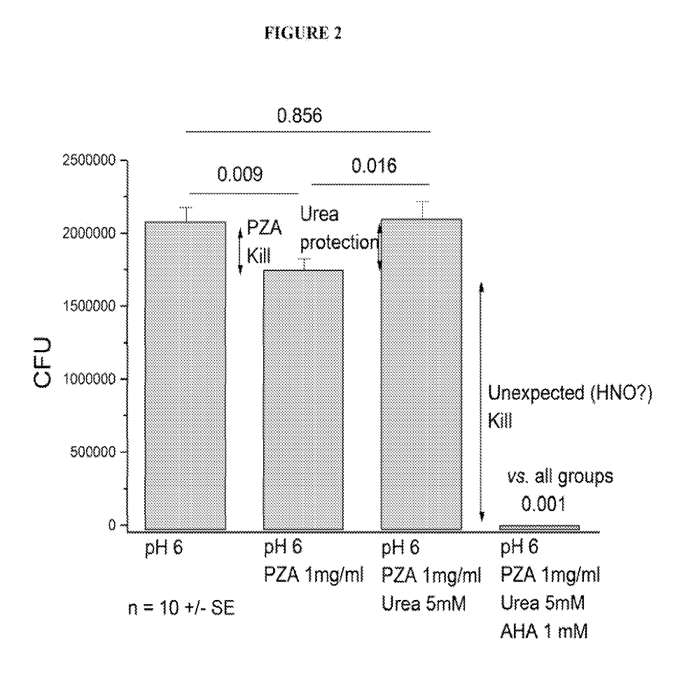

We employed a time-kill curve method to determine the bactericidal activity of PZA and a combination of PZA and acetohydroxamic acid (AHA) against M. tuberculosis. We cultured M. tuberculosis on solid media at pH6. CFU values were determined for samples treated with pyrazinamide (PZA), urea at a typical body fluid concentration and acetohydroxamic acid. As shown in FIG. 2, we determined that the kill by PZA alone was modest, and that urea protected against the PZA kill as expected. Surpassingly, we determined that AHA not only prevented urea protection, it also substantially increased the kill rate. These results demonstrate activation of AHA by TB enzymes to produce nitroxyl (HNO), a very damaging species. Consequently, our results provide that AHA acts not only as a PZA booster, but also as an anti-mycobacterial agent, in some instances, in a synergistic manner with the traditional anti-mycobacterial agent used.

1. A method of treating a subject who suffers from, or who is suspected of suffering from, a Mycobacterium infection, the method comprising administering to the subject a therapeutically effective amount of a urease inhibitor, optionally in combination with one or more additional anti-mycobacterial agents.

2. The method of claim 1, wherein the Mycobacterium infection is a Mycobacterium Tuberculosis (Mtb) infection, a latent tuberculosis infection (LTBI) or a multidrug-resistant TB (MDR-TB) infection.

3. The method of claim 1, wherein the urease inhibitor is acetohydroxamic acid or a pharmaceutically acceptable salt or derivative thereof.

4. The method of claims 1-3, wherein the one or more additional anti-mycobacterial agents are selected from the group consisting of pyrazinamide, pyrazinoic acid, isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin, ¹⁵N-ethionamide; ³³S-ethionamide, ³⁴S-ethionamide and ³⁶S-ethionamide; and ¹⁵N-isoniazid and pharmaceutical salts and mixtures thereof.

5. The method of claim 1, wherein the subject suffers from, or is suspected of suffering from, a Mycobacterium Tuberculosis (Mtb) infection and is co-administered a therapeutically effective amount of pyrazinamide and/or pyrazinoic acid and acetohydroxamic acid or a pharmaceutically acceptable salt or derivative thereof.

6. (canceled)

7. (canceled)

8. (canceled)

9. (canceled)

10. (canceled)

11. (canceled)

12. A method of preventing a subject's latent Mycobacterium infection from progressing to an active Mycobacterium infection, the method comprising administering to the subject a therapeutically effective amount of a urease inhibitor, optionally in combination with one or more anti-mycobacterial agents.

13. The method of claim 12, wherein the urease inhibitor is acetohydroxamic acid or a pharmaceutically acceptable salt or derivative thereof.

14. The method of claim 12, wherein the one or more anti-mycobacterial agents are selected from the group consisting of pyrazinamide, pyrazinoic acid, isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin and pharmaceutical salts and mixtures thereof.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A pharmaceutical formulation which can be administered by intratracheal instillation, bronchial instillation, or inhalation, or by an oral, intravenous, intramuscular, intra-arterial, intramedullary, subcutaneous, intraventricular, transdermal, interdermal, rectal, intravaginal, intraperitoneal, topical, transdermal, mucosal, nasal, buccal, enteral, or sublingual route of administration, the formulation comprising:
(a) an amount of a urease inhibitor which is therapeutically effective in reducing the likelihood of the onset of or treating a Mycobacterium infection;
(b) optionally one or more anti-mycobacterial agents selected from the group consisting of pyrazinamide, pyrazinoic acid, isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin, ¹⁵N-ethionamide; ³³S-ethionamide, ³⁴S-ethionamide, ³⁶S-ethionamide; ¹⁵N-isoniazid, a compound of the Formula (II): 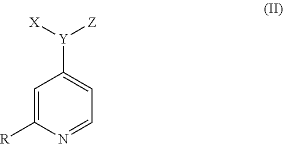
where X is an oxygen atom selected from the group consisting of ¹⁷O and ¹⁸O; Y is a carbon atom selected from the group consisting of ¹²C and ¹³C; Z is a NHNH₂ group, which group is optionally isotopically labeled with at least one ¹⁵N atom; and R is H, and mixtures thereof or an analog, derivative, pharmaceutically acceptable salt, enantiomer, diastereomer, solvate or polymorph thereof; and
(c) one or more pharmaceutically acceptable excipients.

35. (canceled)

36. An inhalable dry powder pharmaceutical formulation comprising:
(a) an amount of a urease inhibitor which is therapeutically effective in treating a Mycobacterium infection;
(b) optionally, one or more anti-mycobacterial agents selected from the group consisting of pyrazinamide, pyrazinoic acid, isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin, ¹⁵N-ethionamide; ³³S-ethionamide, ³⁴S-ethionamide, ³⁶S-ethionamide; ¹⁵N-isoniazid, a compound of the Formula (II): 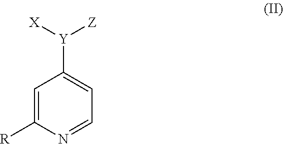
where X is an oxygen atom selected from the group consisting of ¹⁷O and ¹⁸O; Y is a carbon atom selected from the group consisting of ¹²C and ¹³C; Z is a NHNH₂ group, which group is optionally isotopically labeled with at least one ¹⁵N atom; and R is H, and mixtures thereof or an analog, derivative, pharmaceutically acceptable salt, enantiomer, diastereomer, solvate or polymorph thereof; and
(c) particles of a physiologically acceptable pharmacologically-inert solid carrier.

37. A dry powder inhaler comprising the inhalable dry powder formulation of claim 36.

38.-49. (canceled)

50. The method of claim 13, wherein the one or more anti-mycobacterial agents are selected from the group consisting of pyrazinamide, pyrazinoic acid, isoniazid, ethionamide, aminosalicyclic acid/aminosalicylate sodium, capreomycin sulfate, clofazimine, cycloserine, ethambutol hydrochloride, kanamycin sulfate, rifabutin, rifampin, rifapentine, streptomycin sulfate, gatifloxacin and pharmaceutical salts and mixtures thereof.
